(19) United States
(12) Patent Application Publication
(10) Pub. No.: US 2018/0149658 A1
(43) Pub. Date: May 31, 2018

(54) METHODS AND KITS FOR CANCER ANTIGEN AND HEPARAN SULFATE IMAGING

(71) Applicant: Bio-Techne Corporation, Minneapolis, MN (US)

(72) Inventors: Zhengliang L. Wu, Edina, MN (US); Anthony D. Person, Blaine, MN (US)

(21) Appl. No.: 15/826,764

(22) Filed: Nov. 30, 2017

Related U.S. Application Data
(60) Provisional application No. 62/428,216, filed on Nov. 30, 2016.

(51) Int. Cl. G01N 33/58; C07K 14/47; C07K 16/28; C12P 21/00; C12Q 1/48

(52) U.S. Cl. CPC G01N 33/582; C07K 14/4748; C07K 16/2866; C12P 21/005; G01N 2440/38; C07K 2317/41; G01N 2333/91091; G01N 2333/924; G01N 2400/02; C12Q 1/48

(57) ABSTRACT

An in vitro method of imaging a cancer antigen includes providing a cell sample including a cancer antigen selected from the group consisting of a Tn antigen, a T antigen, a sialylated-Tn antigen, and a sialylated-T antigen, treating the sample with a glycosyltransferase to incorporate a carbohydrate with a click chemistry moiety into the cancer antigen, adding a label to the sample that includes a click chemistry moiety that reacts to the click chemistry moiety of the carbohydrate such that the label attaches to the carbohydrate to form a labeled cancer antigen, and imaging the sample with a camera.

### CROSS-REFERENCE

This application claims priority to U.S. Provisional Application No. 62/428,216, filed Nov. 30, 2016, the entire contents of which are incorporated herein by reference.

### TECHNICAL FIELD

This disclosure relates to glycans, and more particularly, to methods and kits for imaging glycans.

### BACKGROUND

Glycans, together with nucleic acids, proteins and lipids, are basic molecules for all living organisms. Glycosylation is the most common type of post-translational modification. Glycans are assembled by various glycosyltransferases along the pathway from the endoplasmic reticulum to the Golgi apparatus until they are finally secreted to the cellular membrane or extracellular matrix. Glycans play various biological roles from protein folding and quality control to a large number of biological recognition events, such as acting as receptors to numerous glycan-binding lectins, growth factors, and cytokines.

Glycans usually are displayed on the cell surface and in the extracellular matrix in the forms of N-glycans, O-glycans and glycosaminoglycans. A representative O-glycan is core-1 O-glycan (Galα1-3GalNAc-R), also known as a T antigen, which is displayed on various cancer cells. T antigen synthesis occurs by adding a GalNAc residue and a Gal residue to a protein peptide using glycosyltransferases specific to the GalNAc and Gal residues. Loss of activity of a glycosyltransferase during T antigen synthesis can result in an intermediate product, O-GalNAc, also known as a Tn antigen. Additionally, sialylation of T antigens can result in formation of sialylated-T antigens (NeuNAc-(2-3)-βGal-(1-3)-αGalNAc-O-Ser/Thr). T antigens, Tn antigens, and sialylated-T antigens are hallmarks of cancer etiology. For example, sialylated-T antigens have been identified as the most abundant glycan found in different tumor cell lines, such as the breast cancer cell line T47D and the gastric carcinoma cell lines HT-29 and K562.

A representative glycosaminoglycan is heparan sulfate (HS), a linear polysaccharide that has repetitive disaccharide units of HexA-GlcNAc. HS is synthesized by EXTs, dual enzymes that exhibit both GlcA and GlcNAc transferase activities. HS can be degraded by heparanase (HPSE), an endoglucuronidase that specifically hydrolyzes the GlcA-GlcNAc bond in highly sulfated HS domains.

Despite the abundance of glycans and their important biological functions, methods for synthesis of glycan cancer markers are usually based on chemical or chemoenzymatic methods. Such methods are inefficient and require the use of organic solvents and hazardous materials. Furthermore, glycans are notoriously difficult to image, due to a lack of high affinity antibodies or binding proteins. Current imaging techniques rely on antibodies or plant lectins. Anti-glycan antibodies are rare, and it is hard to determine their specificities. Lectins generally have low affinity binding sites. Use of antibodies or lectins can lead to unreliable and inaccurate results due to labeling of unwanted structures in addition to the target glycans. Therefore, antibodies and lectins are not ideal for glycan imaging on cells.

### SUMMARY

In general, this disclosure relates to methods and kits for imaging glycans, including O-glycans and glycosaminoglycans. For O-glycan synthesis and imaging, using an enzymatic method, Tn, T, and sialylated-T antigens are synthesized on cells in vitro or on proteins in an artificial scaffold. The synthesized cancer antigens are then prepared for imaging through incorporation of clickable carbohydrates using specific glycosyltransferases, followed by the addition of fluorescent or colorimetric labels through a click chemistry reaction to form labeled cancer antigens. The labeled cancer antigen is subsequently imaged with a camera. For imaging naturally occurring Tn and T antigens on cells in vitro, the cells are first treated with glycosidase, such as sialidase (neuraminidase), to remove the terminal sialic acid from the antigens and expose glycosyltransferase recognition sites. The treated cells are then prepared for imaging through incorporation of clickable carbohydrates using specific glycosyltransferases, followed by the addition of fluorescent or colorimetric labels through a click chemistry reaction to form labeled cancer antigens. The labeled cancer antigen is subsequently imaged with a camera.

For glycosaminoglycan imaging, in vitro heparan sulfate chains are treated with a glycosyltransferase to incorporate a clickable carbohydrate into the non-reducing end of each heparan sulfate chain. Fluorescent or colorimetric labels are then added through a click chemistry reaction to form labeled heparan sulfate chains, which are subsequently imaged with a camera. Since the glycosyltransferases are specific to substrate glycans, these methods are highly specific. Furthermore, the labels are attached to the target glycans via covalent bonding, which eliminates the lack of specificity and sensitivity seen in imaging techniques that rely on low affinity binding.

In one embodiment, an in vitro method of imaging a cancer antigen includes providing a sample including a cancer antigen selected from the group consisting of a Tn antigen, a T antigen, a sialylated-Tn antigen, and a sialylated-T antigen, treating the sample with a glycosyltransferase to incorporate a carbohydrate with a click chemistry moiety into the cancer antigen, adding a label to the sample that includes a click chemistry moiety that reacts to the click chemistry moiety of the carbohydrate such that the label attaches to the carbohydrate to form a labeled cancer antigen, and imaging the sample with a camera.

In another embodiment, an in vitro method of synthesizing and imaging a Tn antigen includes providing a sample including a polypeptide chain having a serine or threonine residue, treating the sample with a first glycosyltransferase to attach a GalNAc residue to the serine or threonine residue, treating the sample with a second glycosyltransferase to incorporate a carbohydrate with a click chemistry moiety into the GalNAc residue, adding a label to the sample that includes a click chemistry moiety that reacts to the click chemistry moiety of the carbohydrate such that the label attaches to the carbohydrate to form a labeled Tn antigen, and imaging the sample with a camera.

In another embodiment, an in vitro method of synthesizing and imaging a T antigen includes providing a sample comprising a polypeptide chain having a serine or threonine residue, treating the sample with a first glycosyltransferase to attach a GalNAc residue to the serine or threonine residue, treating the sample with a second glycosyltransferase to attach a galactose residue to the GalNAc residue, treating the sample with a third glycosyltransferase to incorporate a carbohydrate with a click chemistry moiety into the GalNAc residue or the galactose residue, adding a label to the sample that includes a click chemistry moiety that reacts to the click chemistry moiety of the carbohydrate such that the label attaches to the carbohydrate to form a labeled T antigen, and imaging the sample with a camera.

In another embodiment, an in vitro method of synthesizing and imaging a sialylated-T antigen includes providing a sample comprising a polypeptide chain having a serine or threonine residue, treating the sample with a first glycosyltransferase to attach a GalNAc residue to the serine or threonine residue, treating the sample with a second glycosyltransferase to attach a galactose residue to the GalNAc residue, treating the sample with a third glycosyltransferase to attach a sialic acid residue to the galactose residue, treating the sample with a fourth glycosyltransferase to incorporate a carbohydrate with a click chemistry moiety into the GalNAc residue, adding a label to the sample that includes a click chemistry moiety that reacts to the click chemistry moiety of the carbohydrate such that the label attaches to the carbohydrate to form a labeled sialylated-T antigen, and imaging the sample with a camera.

In another embodiment, an in vitro method of imaging heparan sulfate includes providing a sample comprising a heparan sulfate chain having an extendable non-reducing end, treating the sample with a glycosyltransferase to incorporate a carbohydrate with a click chemistry moiety into the extendable non-reducing end of the heparan sulfate chain, adding a label to the sample, wherein the label includes a click chemistry moiety that reacts to the click chemistry moiety of the carbohydrate such that the label attaches to the carbohydrate to form a labeled heparan sulfate chain, and imaging the sample with a camera.

In another embodiment, a kit for imaging a cancer antigen in vitro includes a cancer antigen selected from the group consisting of a Tn antigen, a T antigen, a sialylated-Tn antigen, and a sialylated-T antigen, a glycosyltransferase, a donor carbohydrate with a click chemistry moiety, a label including a click chemistry moiety that reacts to the click chemistry moiety of the donor carbohydrate, and click chemistry reagents.

In another embodiment, an in vitro method of screening a test substance as a therapeutic agent for treating cancer includes providing a sample comprising a cancer cell, treating the sample with the test substance, treating the sample with a glycosyltransferase to incorporate a carbohydrate with a click chemistry moiety into a cancer antigen selected from the group consisting of a Tn antigen, a T antigen, a sialylated-Tn antigen, and a sialylated-T antigen, adding a label to the sample that includes a click chemistry moiety that reacts to the click chemistry moiety of the carbohydrate such that the label attaches to the carbohydrate to form a labeled cancer antigen that generates a signal upon imaging, imaging the sample with a camera to determine the strength of the signal generated by the labeled cancer antigen, comparing the image of the sample treated with the test substance with an image of an untreated sample comprising a cancer cell with the labeled cancer antigen, and designating the test substance as a therapeutic agent for treating cancer when the signal generated by the labeled cancer antigen of the sample treated with the test substance is weaker than the signal generated by the labeled cancer antigen in the untreated sample.

In another embodiment, a kit for imaging heparan sulfate in vitro includes a heparan sulfate chain having a non-reducing end, a glycosyltransferase, a donor carbohydrate with a click chemistry moiety, a label including a click chemistry moiety that reacts to the click chemistry moiety of the donor carbohydrate, and click chemistry reagents.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a flow diagram of a method of imaging a cancer antigen according to various embodiments.

FIG. 2 is a flow diagram of methods of synthesizing cancer antigens for imaging according to various embodiments.

FIG. 3 is a flow diagram of methods of attaching a label to a sample for imaging according to various embodiments.

FIG. 4 is a flow diagram of a method of imaging a heparan sulfate chain according to various embodiments.

FIG. 5 is a flow diagram of methods of preparing heparan sulfate chains for imaging according to various embodiments.

FIG. 6 is an image of C3H/10T1/2 cells on which Tn antigens were synthesized and imaged.

FIG. 7 is an image of C3H/10T1/2 cells on which T antigens were synthesized and imaged.

FIG. 8 is an image of HUVEC cells on which Tn antigens were synthesized and imaged.

FIG. 9 is an image of HUVEC cells on which heparan sulfate was imaged.

FIG. 10 is an image of HUVEC cells imaged for heparan sulfate after digestion with heparinase III.

FIG. 11 is an image of HUVEC cells imaged for heparan sulfate after digestion with heparanase.

FIG. 12 is an image of HUVEC cells imaged for heparan sulfate without UDP-GlcA after digestion with heparanase.

FIG. 13 is an image of HUVEC cells on which T antigens were synthesized and imaged.

FIG. 14 is an image of HUVEC cells on which Tn antigens were synthesized but T antigens were imaged.

FIG. 15 is an image of HUVEC cells on which Tn antigens were synthesized and imaged.

FIG. 16 is an image of HUVEC cells on which T antigens were synthesized and imaged.

FIG. 17 is an image of HUVEC cells on which sialyalted-T antigens were synthesized and imaged.

FIG. 18 is an image of HeLa cells containing T, Tn, sialylated-T, and sialylated-Tn antigens on which the antigens were imaged.

FIG. 19 is an image of HeLa cells containing T, Tn, sialylated-T, and sialylated-Tn antigens on which the antigens and the cell nuclei were imaged.

### DETAILED DESCRIPTION

The following detailed description is exemplary in nature and is not intended to limit the scope, applicability, or configuration of the disclosure in any way. Unless defined otherwise, all technical and scientific terms used herein have the same meaning as those commonly understood by one of ordinary skill in the art to which this invention belongs. Although methods and materials similar or equivalent to those described herein may be used in the invention or testing, suitable methods and materials are described herein. The materials, methods and examples are illustrative only, and are not intended to be limiting. Those skilled in the art will recognize that many of the noted examples have a variety of suitable alternatives. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

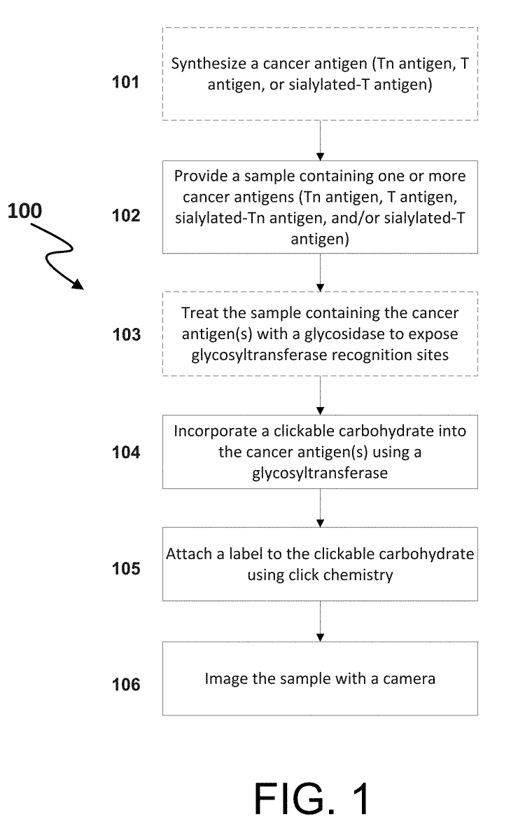

FIG. 1 is a flow diagram of method 100 of imaging a cancer antigen in vitro. Method 100 can include synthesizing a cancer antigen (101), providing a sample containing one or more cancer antigens (102), treating the sample containing the cancer antigen(s) with a glycosidase to expose glycosyltransferase recognition sites (103), incorporating a clickable carbohydrate into the cancer antigen(s) using a glycosyltransferase (104), attaching a label to the clickable carbohydrate using click chemistry (105), and imaging the sample with a camera (106). Method 100 need not include all of the steps shown in FIG. 1. For example, in some embodiments, method 100 may exclude the step of synthesizing a cancer antigen (101) and/or the step of treating the sample containing the cancer antigen(s) with a glycosidase (103).

Method 100 allows for imaging cancer antigens, specifically Tn antigens, T antigens, sialylated-Tn antigens, and sialylated-T antigens, by using click chemistry to label the target antigens. In order to perform method 100, a cancer antigen can first be synthesized on a cell sample in vitro (101) or an in vitro cell sample containing the desired cancer antigen for imaging can be provided (102). In one embodiment, an in vitro cell sample is provided (102) containing naturally occurring cancer antigens. In that embodiment, the sample can be treated with a glycosidase to expose glycosyltransferase recognition sites (103) on the naturally occurring cancer antigens. In one example, the glycosidase is sialidase and is used to treat the sample to remove the terminal sialic acid from sialylated-T antigens and/or sialylated-Tn antigens. An in vitro cell sample can be provided that contains one or more naturally occurring cancer antigens, specifically Tn antigens, T antigens, sialylated-Tn antigens, and sialylated-T antigens, and method 100 can be used to image some or all of those antigens. If a glycosidase such as sialidase is used remove terminal sialic acids and expose glycosyltransferase recognition sites, all of the Tn antigens, T antigens, sialylated-Tn antigens, and sialylated-T antigens in a sample can be labeled and imaged.

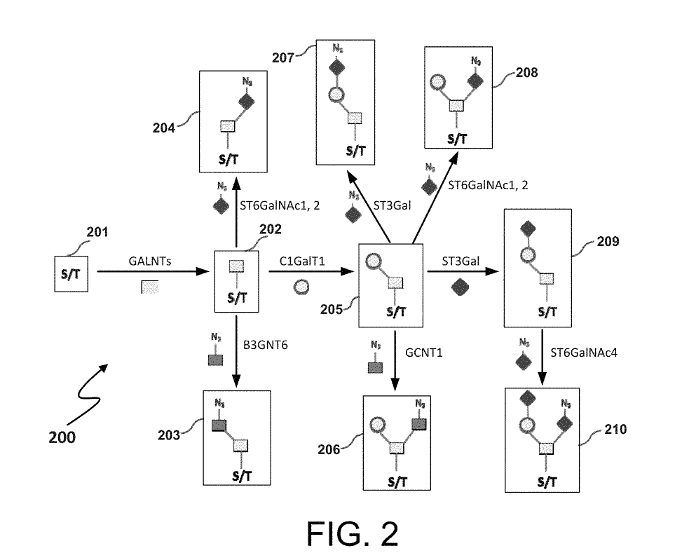

In some embodiments, the cancer antigen can be synthesized or provided on proteins in an artificial scaffold. In some embodiments, human umbilical vein endothelial cell (HUVEC) samples can be used. HUVEC cells are commonly used to study the mechanisms of angiogenesis in vitro. In other embodiments, HeLa cells can be used. HeLa cells are derived from cervical cancer cells and are commonly used in research. In other embodiments, mesenchymal stem cell samples can be used. Mesenchymal stem cells are able to develop into tissues of the lymphatic and circulatory systems, as well as connective tissues throughout the body, such as bone and cartilage. The synthesis of Tn antigens, T antigens, and sialylated-T antigens is described in greater detail with respect to FIG. 2 below. It has been discovered that clickable carbohydrates can be incorporated into Tn antigens, T antigens, sialylated-Tn antigens, and sialylated-T antigens, thus allowing for labeling of the antigens for imaging.

In order to incorporate a clickable carbohydrate into a cancer antigen, the cell sample containing the target antigen is treated with a glycosyltransferase specific to the cancer antigen (104). In some embodiments, the glycosyltransferase can be a recombinant glycosyltransferase. In some embodiments, the glycosyltransferase can be a beta-1,3-N-acetylglucosaminyltransferases such as B3GNT6, a beta-1,6 N-acetylglucosaminyltransferase such as GCNT1, a sialyltransferase such as ST3Gal1 or ST3Gal2, a ST6 N-acetylgalactosaminide alpha-2,6-sialyltransferase, such as ST6GalNAc1, ST6GalNAc2, or ST6GalNAc4, or combinations thereof. The clickable carbohydrate includes a click chemistry moiety that can be used in a click chemistry reaction, such as an azido or alkyne group. In some cases, the carbohydrate is a monosaccharide. It has been discovered that azidoacetylglucosamine (GlcNAz), which includes an azido group, is a suitable clickable carbohydrate for incorporation into cancer antigens, particularly Tn antigens and T antigens. It has also been discovered that azido-sialic acid, which includes an azido group, is also a suitable clickable carbohydrate for incorporation into cancer antigens, particularly T antigens, sialylated-T antigens, and Tn antigens.

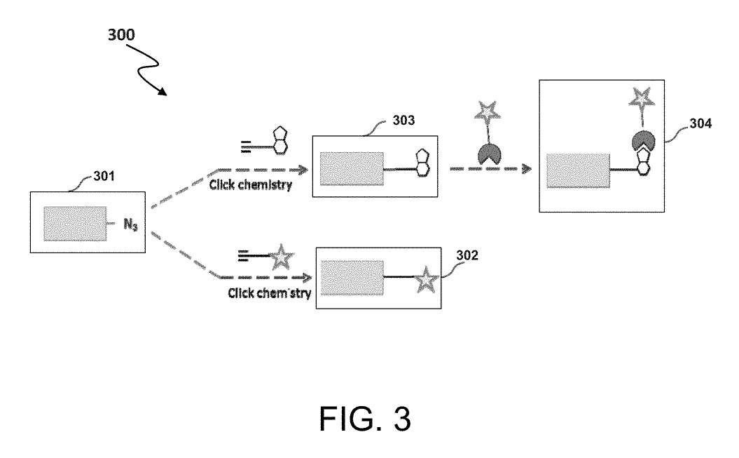

Once the clickable carbohydrate is incorporated into the target antigen, a label is attached to the clickable carbohydrate on the target antigen through click chemistry (105). Click chemistry is a way to quickly and reliably join small units together. It is not a single specific reaction, but refers to a general way of joining small modular units. The label includes a click chemistry moiety that reacts to the click chemistry moiety of the incorporated carbohydrate such that the label attaches to the carbohydrate. In some embodiments, the carbohydrate includes an azido group and the label includes an alkyne group. In other embodiments, the carbohydrate includes an alkyne group and the label includes an azido group. The clickable label can be a reporter molecule, such as a fluorescent label, a colorimetric label, a biotin conjugate linked to a fluorescent label, or a biotin conjugate linked to a colorimetric label. The attachment of the label to the cancer antigen containing the clickable carbohydrate is described in greater detail with respect to FIG. 3 below.

Upon attachment of the label to the cancer antigen, the labeled antigen can be imaged using a camera suitable for detecting the specific label (106). In some embodiments, the camera can be a fluorescent camera or a colorimetric camera. Images produced using method 100 show the location within the cell, as well as the abundance of the target antigen, which provides valuable insight into cancer etiology and treatment. Method 100 is also advantageous, because it is highly specific and thus eliminates specificity and sensitivity issues that can arise from using antibodies or lectins for glycan imaging. The specificity of method 100 is due to the use of glycosyltransferases that are highly specific to the target antigens. The sensitivity of method 100 is due to the use of covalent conjugation of the labels to the target antigens, which eliminates any affinity issues that can arise. Covalently linked molecules will stay bound under extreme conditions without losing their bond while non-covalently linked molecules can detach under harsh conditions such as low pH and high salt concentrations. For example, non-covalently linked labels detach under stringent washing conditions used in lectin binding assays. This results in a lost or weakened signal.

Method 100 can be used to screen potential therapeutic agents for treating cancer. Since antigens such as Tn antigens and T antigens are hallmarks of cancer, cancer cells can be treated with a potential therapeutic agent to determine if such treatment reduces the presence of cancer antigens in those cancer cells. For example, cancer cells can be treated with a potential therapeutic agent and the antigens in the cancer cells subsequently labeled and imaged using method 100 to determine the strength of the signal generated by the labeled cancer antigens within the treated cancer cells. The image of the treated cancer cells can be compared to an image of untreated cancer cells also labeled and imaged using method 100. If the signal generated by the labeled cancer cells treated with the potential therapeutic agent is weaker than the signal generated by the untreated labeled cancer cells, the potential therapeutic agent can be designated a therapeutic agent for treating cancer.

FIG. 2 is a flow diagram of method 200 of synthesizing cancer antigens, according to various embodiments. Method 200 corresponds to step 101 of method 100, which is described with respect to FIG. 1 above. Method 200 is a purely enzymatic method that can be used to synthesize T antigens, Tn antigens, and sialylated-T antigens. To form a cancer antigen, a cell sample or a protein polypeptide is provided having an unmodified serine or threonine residue (201). In some embodiments, the serine or threonine residue is located on a cell membrane protein. In other embodiments, the serine or threonine residue is located on a protein in the extracellular matrix. In other embodiments, the serine or threonine residue is located on a protein in an artificial scaffold.

To form a Tn antigen, the cell sample is treated with a glycosyltransferase to attach a GalNAc residue to the serine or threonine residue (202). The glycosyltransferase recognizes the serine or threonine residue as a carbohydrate attaching site. In some embodiments, the glycosyltransferase can be a recombinant glycosyltransferase. In some embodiments, the glycosyltransferase can be polypeptide N-acetylgalactosaminyl-transferase such as GALNT1, GALNT2, GALNT3, or mixtures thereof.

In order to prepare the Tn antigen for imaging, the cell sample is treated with a glycosyltransferase to incorporate a clickable carbohydrate into the GalNAc residue (203, 204). In some embodiments, the glycosyltransferase can be a recombinant glycosyltransferase. In some embodiments, the glycosyltransferase can be a beta-1,3-N-acetylglucosaminyltransferase such as B3GNT6. In some cases, the carbohydrate is a monosaccharide. It has been discovered that GlcNAz is a suitable clickable carbohydrate for incorporation into Tn antigens. In one embodiment, the cell sample is treated with B3GNT6 to incorporate GlcNAz into the Tn antigen (203). In other embodiments, the glycosyltransferase can be a ST6 N-acetylgalactosaminide alpha-2,6-sialyltransferase, such as ST6GalNAc1 or ST6GalNAc2. It has been discovered that azido-sialic acid is also a suitable clickable carbohydrate for incorporation into Tn antigens. In one embodiment, the cell sample is treated with ST6GalNAc 1 or 2 to incorporate azido-sialic acid into the Tn antigen (204).

Once the clickable carbohydrate is incorporated into the Tn antigen, a label is attached to the clickable carbohydrate on the target antigen through click chemistry. The clickable label can be a reporter molecule, such as a fluorescent label, a colorimetric label, a biotin linked to a fluorescent label, or a biotin linked to a colorimetric label. The attachment of the label to the cancer antigen containing the clickable carbohydrate is described in greater detail with respect to FIG. 3 below.

To form a T antigen, a cell sample with a Tn antigen (202) is treated with a glycosyltransferase to attach a galactose residue to the GalNAc residue on the Tn antigen (205). In some embodiments, the glycosyltransferase can be a recombinant glycosyltransferase. In some embodiments, the glycosyltransferase can be a core 1 beta-3-galactosyltransferase such as C1GalT1.

In order to prepare the T antigen for imaging, in one embodiment, the cell sample is subsequently treated with a glycosyltransferase to incorporate a clickable carbohydrate into the T antigen by attaching the clickable carbohydrate to the GalNAc residue (206, 208). In some embodiments, the glycosyltransferase can be a recombinant glycosyltransferase. In some embodiments, the glycosyltransferase can be a beta-1,6 N-acetylglucosaminyltransferase such as GCNT1. In some cases, the carbohydrate is a monosaccharide. It has been discovered that GlcNAz is a suitable clickable carbohydrate for incorporation into T antigens, specifically when attached to the GalNAc residue. In one embodiment, the cell sample is treated with GCNT1 to incorporate GlcNAz into the T antigen (206). In other embodiments, the glycosyltransferase can be a ST6 N-acetylgalactosaminide alpha-2,6-sialyltransferase, such as ST6GalNAc1 or ST6GalNAc2. It has been discovered that azido-sialic acid is also a suitable clickable carbohydrate for incorporation into Tn antigens. In one embodiment, the cell sample is treated with ST6GalNAc 1 or 2 to incorporate azido-sialic acid into the T antigen (208).

In another embodiment, the cell sample is subsequently treated with glycosyltransferase to incorporate a clickable carbohydrate into the T antigen by attaching the clickable carbohydrate to the galactose residue (207). In some embodiments, the glycosyltransferase can be a recombinant glycosyltransferase. In some embodiments, the glycosyltransferase can be a ST3 beta-galactoside alpha-2,3-sialyltransferase such as ST3Gal1, ST3Gal2, or mixtures thereof. In some cases, the carbohydrate is a monosaccharide. It has been discovered that azido-sialic acid is a suitable clickable carbohydrate for incorporation into T antigens, specifically when attached to the galactose residue. In one embodiment, the cell is treated with ST3Gal 1 or 2 to incorporate azido-sialic acid into the T antigen (207).

Once the clickable carbohydrate is incorporated into the T antigen, a label is attached to the clickable carbohydrate on the target antigen through click chemistry. The clickable label can be a reporter molecule, such as a fluorescent label, a colorimetric label, a biotin linked to a fluorescent label, or a biotin linked to a colorimetric label. The attachment of the label to the cancer antigen containing the clickable carbohydrate is described in greater detail with respect to FIG. 3 below.

To form a sialylated-T antigen, a cell sample with a T antigen (205) is treated with a glycosyltransferase to attach a sialic acid residue to the Galactose residue on the T antigen (209). In some embodiments, the glycosyltransferase can be a recombinant glycosyltransferase. In some embodiments, the glycosyltransferase can be a ST3 beta-galactoside alpha-2,3-sialyltransferases such as ST3Gal1, ST3Gal2, or mixtures thereof

In order to prepare the sialylated-T antigen for imaging, the cell sample is subsequently treated with a glycosyltransferase to incorporate a clickable carbohydrate into the GalNAc residue (210). In some embodiments, the glycosyltransferase can be a recombinant glycosyltransferase. In some embodiments, the glycosyltransferase can be a ST6 N-acetylgalactosaminide alpha-2,6-sialyltransferase such as ST6GalNAc1, ST6GalNAc2, ST6GalNAc4, or mixtures thereof. It has been discovered that azido-sialic acid is a suitable clickable carbohydrate for incorporation into sialylated-T antigens. In one embodiment, the cell sample is treated with ST6GalNac4 to incorporate azido-sialic acid into the sialylated-T antigen (210).

Once the clickable carbohydrate is incorporated into the sialylated-T antigen, a label is attached to the clickable carbohydrate on the target antigen through click chemistry. The clickable label can be a reporter molecule, such as a fluorescent label, a colorimetric label, a biotin linked to a fluorescent label, or a biotin linked to a colorimetric label. The attachment of the label to the cancer antigen containing the clickable carbohydrate is described in greater detail with respect to FIG. 3 below.

The synthesis of Tn, T, and sialylated-T antigens in method 200 is purely enzymatic. Method 200 is thus advantageous, because it is highly efficient and eliminates the need to use organic solvents and hazardous materials to synthesize cancer antigens.

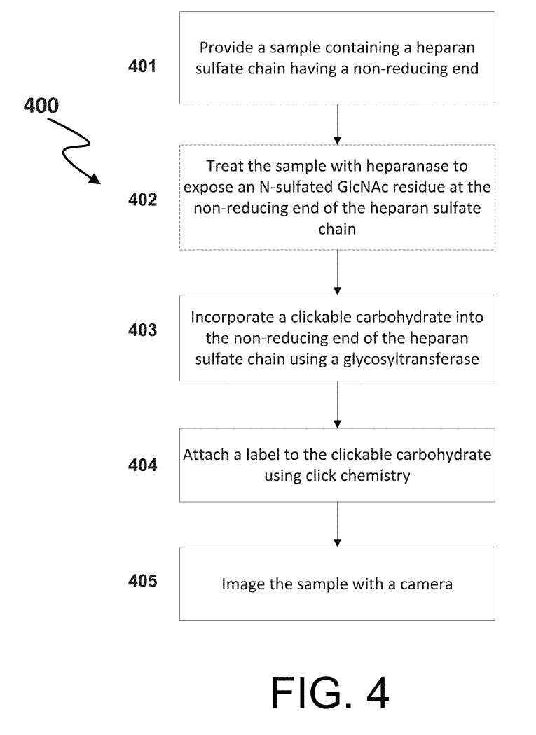

FIG. 3 is a flow diagram of method 300 of attaching a label to a target substrate in a sample, such as a cancer antigen or heparan sulfate chain, for imaging according to various embodiments. Method 300 corresponds to step 104 in method 100, which is described with respect to FIG. 1 above. Method 300 also corresponds to step 404 in method 400, which is described with respect to FIG. 4 below.

Once a clickable carbohydrate is attached to a target substrate (301), a label is attached to the clickable carbohydrate via click chemistry in order to be able to image the target substrate. The label includes a click chemistry moiety that reacts to the click chemistry moiety of the carbohydrate such that the label attaches to the carbohydrate. In some embodiments, the clickable carbohydrate includes an alkyne group click chemistry moiety and the label includes an azido group click chemistry moiety. In the embodiments shown in FIG. 3, the clickable carbohydrate includes an azido click chemistry moiety and the label includes an alkyne group click chemistry moiety.

In some embodiments, the label can be a fluorescent molecule or a colorimetric molecule. In these embodiments, the fluorescent or colorimetric molecule directly attaches to the clickable carbohydrate on the target substrate via click chemistry to form a labeled target substrate (302). In other embodiments, the label can be a biotin linked to a fluorescent label, such as Alexa Fluor® 555 or Alexa Fluor® 488, or a colorimetric label. In these embodiments, a biotin is first attached to the clickable carbohydrate on the target substrate via click chemistry to form a biotinylated target substrate (303). A fluorescent label, such as Alexa Fluor® 555 or Alexa Fluor® 488, or a colorimetric label attached to a streptavidin is subsequently attached to the biotin on the target substrate to form a labeled target substrate (304).

FIG. 4 is a flow diagram of method 400 of imaging a heparan sulfate chain in vitro according to various embodiments. Heparan sulfate (HS) is a linear polysaccharide found in the extracellular matrix and on the cell membrane and plays a role in a number of cellular events, including cell growth, migration, and differentiation. HS binds various growth factors, cytokines, and other extracellular matrix proteins. Heparanase (HPSE) is a hydrolase and the only known enzyme that cleaves heparan sulfate (HS) in the extracellular matrix and cell membrane. HPSE digestion of HS facilitates cell invasion and metastasis of cancer.

Method 400 can include providing a sample containing a heparan sulfate chain having a non-reducing end (401), treating the sample with HPSE to expose an N-sulfated GlcNAc residue at the non-reducing end of the heparan sulfate chain (402), incorporating a clickable carbohydrate into the non-reducing end of the heparan sulfate chain using a glycosyltransferase (403), attaching a label to the clickable carbohydrate using click chemistry (404), and imaging the sample with a camera (405). Method 400 need not include all of the steps shown in FIG. 4. For example, in some embodiments, method 400 may exclude the step of treating the sample with HPSE (402).

Method 400 allows for imaging heparan sulfate by using click chemistry to label the heparan sulfate. In order to perform method 400, an in vitro cell sample containing heparan sulfate can be provided (401). In some embodiments, HUVEC cell samples can be used. In other embodiments, mesenchymal stem cell samples can be used. It has been discovered that clickable carbohydrates can be incorporated into heparan sulfate chains, thus allowing for labeling of the heparan sulfate. In some embodiments, a single clickable carbohydrate is incorporated into each heparan sulfate chain. In other embodiments, multiple clickable carbohydrates, such as two clickable carbohydrates, are incorporated into each heparan sulfate chain.

In order to prepare the heparan sulfate in the cell sample for imaging, a clickable carbohydrate is incorporated into the non-reducing end of the heparan sulfate chain (403). In some embodiments, prior to incorporating a clickable carbohydrate into the heparan sulfate chain, the sample can be treated with HPSE to expose an N-sulfated GlcNAc residue at the non-reducing end of the heparan sulfate chain (402). In these embodiments, the clickable carbohydrate is subsequently incorporated into the N-sulfated GlcNAc residue at the non-reducing end of the heparan sulfate chain.

In order to incorporate a clickable carbohydrate into a heparan sulfate chain, the cell sample containing the heparan sulfate is treated with a glycosyltransferase specific to the heparan sulfate (403). The clickable carbohydrate is thus incorporated into the non-reducing end of each heparan sulfate chain. In some embodiments, the glycosyltransferase can be a recombinant glycosyltransferase. In some embodiments, the glycosyltransferase can be EXT1, EXT2, or an EXT1/2 heterodimer. The clickable carbohydrate includes a click chemistry moiety that can be used in a click chemistry reaction, such as an azido or alkyne group. It has been discovered that GlcNAz is a suitable clickable carbohydrate for incorporation into heparan sulfate chains. Clickable GlcA may also be used for incorporation into heparan sulfate chains.

Once the clickable carbohydrate is incorporated into the heparan sulfate chain, a label is attached to the clickable carbohydrate on the target antigen through click chemistry (404). The label includes a click chemistry moiety that reacts to the click chemistry moiety of the incorporated carbohydrate such that the label attaches to the carbohydrate. In some embodiments, the carbohydrate includes an azido group and the label includes an alkyne group. In other embodiments, the carbohydrate includes an alkyne group and the label includes an azido group. The clickable label can be a reporter molecule, such as a fluorescent label, a colorimetric label, a biotin linked to a fluorescent label, or a biotin linked to a colorimetric label. The attachment of the label to the heparan sulfate chain containing the clickable carbohydrate is described in greater detail with respect to FIG. 3 above.

Upon attachment of the label to the heparan sulfate chain, the labeled heparan sulfate can be imaged using a camera suitable for detecting the specific label (405). In some embodiments, the camera can be a fluorescent camera or a colorimetric camera. Images produced using method 400 show the location within the cell, as well as the abundance of heparan sulfate chains, which provides valuable insight into cellular structures and functions. For example, the ability to image heparan sulfate allows for the development of a better understanding of how cells interact with the extracellular matrix. Method 400 is also advantageous, because it is highly specific and thus eliminates specificity issues that can arise from using antibodies or lectins for glycan imaging. The specificity of method 400 is due to the use of glycosyltransferases that are highly specific to heparan sulfate chains. Furthermore, labels are attached to the target antigens via covalent bonding, which eliminates any affinity issues that can arise.

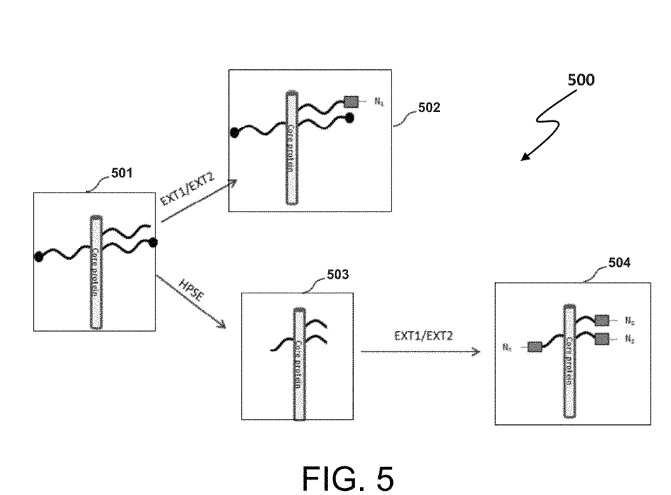

FIG. 5 is a flow diagram of method 500 of preparing heparan sulfate chains for imaging according to various embodiments. Method 500 illustrates steps 402 and 403 of method 400, which is described above with respect to FIG. 4. In order to perform method 500, a cell sample is provided that includes a core protein with heparan sulfate chains (501). In some embodiments, the cell sample can be treated with a glycosyltransferase, such as an EXT1/EXT2 heterodimer to incorporate a clickable carbohydrate into the ends of the heparan sulfate chains (502). In these embodiments, it is likely that the clickable carbohydrate will not be incorporated into all of the heparan sulfate chains in the cell sample. This is because only some non-reducing ends of heparan sulfate chains are extendable (receptive to the incorporation of a clickable carbohydrate).

In other embodiments, the cell sample can be treated with HPSE prior to incorporation of a clickable carbohydrate. HPSE cleaves the non-reducing ends of heparan sulfate chains without completely destroying the heparan sulfate chains. The cleavage of the heparan sulfate chains exposes a portion of the heparan sulfate chain at the non-reducing end that is extendable (receptive to the incorporation of a clickable carbohydrate) (503). This allows for uniformity in the exposed non-reducing ends of the heparan sulfate chains such that a clickable carbohydrate can be attached to substantially all of the heparan sulfate chains in the cell sample (504).

### EXAMPLES

### Materials

CMP-azido-sialic acid, UDP-azido-GalNAc, UDP-GlcNAz (advertised as UDP-azido-GlcNAc), biotinylated alkyne, and 4′,6-diamidino-2-phenylindole (DAPI) were obtained from Bio-Techne®. Recombinant human B4GalT1(Y285L), GALNT2, GCNT1, B3GNT6, EXT1/2, ST6Gal1, ST6GalNAc1, ST6GalNAc2, ST6GalNAc4, C1GalT1 and HPSE, as well as recombinant B. thetaiotaomicron O-GlcNAcase (OGA), recombinant P. heparinus heparinase III (Hep III), and recombinant C. perfringens neuraminidase (sialidase) were also obtained from Bio-Techne®. Streptavidin-Alexa Fluor® 555 and streptavidin-Alexa Fluor® 488 were obtained from Thermo Fisher Scientific®. UDP-Gal and UDP-GlcA and all other small chemicals were from Sigma-Aldrich®.

### Cell Growth and Fixation

C3H10T1/2 cells (CCL-226™ from ATCC®) were grown in MEM NEAA Earle's Salts (Irvine Scientific® Catalog ID: 9130), supplemented with 10% fetal bovine serum (Corning® Catalog #35-015-CV), 2 millimolar (mM) L-glutamine, 100 units/ml penicillin and 0.1 microgram (mg)/milliliter (ml) streptomycin (Sigma-Aldrich® Catalog #G6784). C3H/10T1/2 cells were originally established from cells extracted from C3H mouse embryos and are an established transformed mesenchymal stem cell model, as they can be differentiated into downstream mesenchymal cell lineages. HUVEC cells (Lonza Catalog #C2517A) were grown in an endothelial cell growth medium (Clonetics™ EGM™-2 BulletKit™ from Lonza). Upon confluence, cells were trypsinized and plated in a 24-well cell culture plate and grown to desired confluence. The cells were rinsed with sterile PBS and fixed in 4% paraformaldehyde for 30 minutes at room temperature followed by washing 5 times with sterile phosphate buffered saline (PBS). Upon completion of the washing procedure, the plate was stored in one milliliter sterile PBS at 4 degrees Celsius (° C.) until ready for glycan labeling.

Pretreatment of Cells for Imaging

For glycan imaging, some cells pretreated with glycosidases to remove existing glycans or with glycosyltransferases to synthesize glycans. All of the treatments were applied to cells in a single well of a 24-well plate. To remove sialic acid, for example, cells were treated with 10 micrograms (μg) of recombinant C. perfringens neuraminidase (sialidase) in 200 microliters (μl) of 50 mM Trind 150 mM NaCl at pH 7.5 for 5 minutes at room temperature. Additionally, to remove heparan sulfate, for example, cells were treated with 2 micrograms (μg) of Hep III in 200 microliters (μ1) of a buffer of 50 mM Tris and 2 mM CaCl₂ at pH 7.5 and subsequently incubated for 1 hour at 37° C. To cleave heparan sulfate with HPSE, cells were digested with 2 μg of HPSE in 200 μl of a buffer of 0.1 molar (M) NaOAc at pH 4.0 and subsequently incubated for 1 hour at 37° C. To synthesize Tn antigens on cells, 50 nanomoles (nmol) of UDP-GalNAc and 2 of GALNT2 in 200 μl of a buffer of 25 mMTris, 150 mMNaCl, and 10 mM MnCl2 at pH 7.5 were added into each well. The cells were then incubated at 37° C. for one hour. After either removal or addition of glycans, the cells were washed thoroughly with PBS three times. All solutions were subsequently removed from the samples under vacuum.

### Incorporation of Clickable Carbohydrate on Fixed Cells Using Glycosyltransferases

To incorporate clickable carbohydrates into the target glycans on fixed cells, 25 mM MES, 0.5% (w/v) Triton® X-100, 2.5 mM MgCl₂, 10 mM MnCl₂, 1.25 mM CaCl2 and 0.75 mg/mL of BSA at pH 7.0 was used as a Labeling Buffer. This Labeling Buffer was used for every example described herein. For imaging of Tn antigens, 20 nmol of UDP-GlcNAz and 2 of B3GNT6 were mixed in 200 μl of Labeling Buffer. For imaging of T antigens, 20 nmol of UDP-GlcNAz and 2 μg of GCNT I were mixed in 200 μl of Labeling Buffer. For heparan sulfate imaging, 20 nmol of UDP-GlcNAz, 50 nmol of UDP-GlcA and 4 μg of EXT1/2 were mixed in 200 μl of Labeling Buffer. The mixtures were then applied to wells and incubated at 37° C. for 1 to 2 hours, or at room temperature overnight.

Incorporation of Clickable Carbohydrate on Live Cells Using Glycosyltransferases

To incorporate clickable carbohydrates into the target glycans on live cells, 25 mM Tris, 150 mM NaCl, and 10 mM MnCl2, at pH 7.5 was used as a Labeling Buffer. For imaging T antigens and/or Tn antigens on live HELA cells, 20 nmol of CMP-azido-sialic acid and 2 μg of ST6GalNAc1 were mixed in 200 μl of Labeling Buffer. The mixtures were then applied to wells and incubated at 37° C. for 20 minutes. The labeling mixtures were then removed and the cells were fixed with 5% paraformaldehyde for 10 minutes at room temperature.

### Conjugation of the Clickable Carbohydrate to Biotin and Fluorescent Dye

After the incorporation of the clickable carbohydrate into a target glycan, a biotin moiety was conjugated to the clickable carbohydrate via a click chemistry reaction. For each reaction, 20 nmol of Cu²⁺, 10 nmol of biotinylated alkyne, and 200 nmol of ascorbic acid were combined into a click chemistry mixture having a volume of less than 10 μl in a test tube. The mixture was incubated on a bench top for 1 minute to allow the Cu²⁺ to be reduced to Cu⁺. The mixture was then diluted with 200 μl of a buffer of 25 mM Tris and 150 mM NaCl at pH 7.5. The mixture was subsequently applied to a single well of cells in a 24-well plate and incubated for 30 minutes at room temperature. The click chemistry reaction solution was then removed from the 24-well plate and washed thoroughly with PBS. A fluorescent dye mix of 10 μg/mL streptavidin-Alexa Fluor® 555 or streptavidin-Alexa Fluor® 488 in 200 μL of PBS was then applied to the cells for 15 minutes. In some examples, the fluorescent dye mix also included 10 μM of DAPI in the 200 of PBS. The streptavidin-Alexa Fluor® 555 or streptavidin-Alexa Fluor® 488 bound to the biotin, which resulted in fluorescently labeled target glycans. The cells were then washed thoroughly with PBS and finally stored in PBS.

### Imaging of the Fluorescently Labeled Cells

All images of the labeled cells were captured on an AXIO Observer microscope (ZEISS) with a ZEISS Axiocam 506 mono camera and Zen 2 Pro software. Images were captured simultaneously through the channels of Alexa Fluor® 555 (or Alexa Fluor® 488) and/or DAPI. For most images, the exposure time was automatically set and the contrast was adjusted to be a best fit. For comparison of the results of different pretreatment conditions, such as HPSE pretreatment and Hep III pretreatment for HS staining, the same exposure and contrast adjustment parameters were applied.

### Example 1: Glycan Imaging on C3H/10T1/2 Cells

To demonstrate the method of glycan imaging using in vitro incorporation of clickable monosaccharides, several representative glycans were imaged on C3H/10T1/2 cells. The cells were grown to confluence in a 24-well plate and then probed for Tn antigens and T antigens using B3GNT6 and GCNT1, respectively. B3GNT6 specifically recognizes Tn antigens and synthesizes core-3 O-glycan (GlcNAcβ1-3GalNAcα1-S/T). Therefore B3GNT6 was used for Tn antigen detection in the presence of UDP-GlcNAz. GCNT1 transfers GlcNAc residues to T antigens, and was therefore used for T antigen detection in the presence of UDP-GlcNAz.

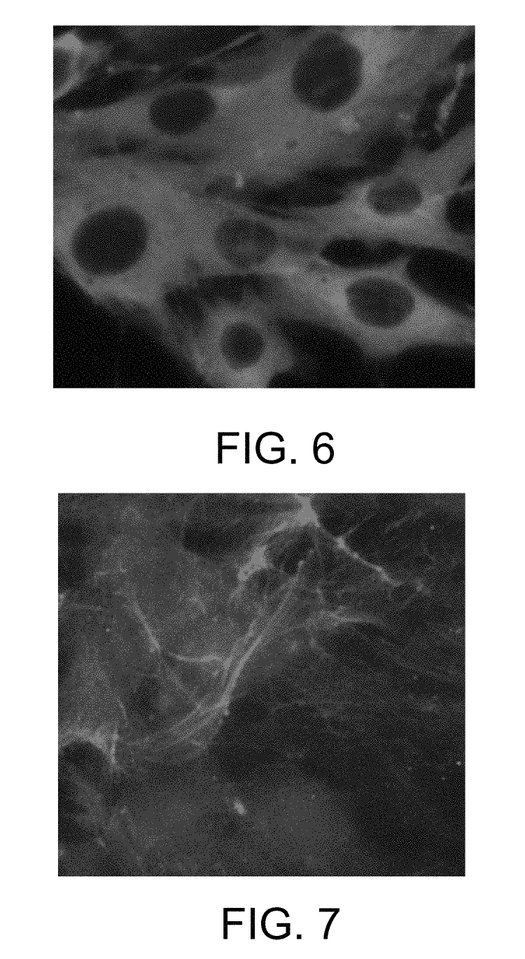

Direct staining for Tn antigens using B3GNT6 resulted in no signal, suggesting that Tn antigens are quickly elongated to more mature O-glycans in vivo. For the purpose of demonstrating Tn antigen staining, Tn antigens were first synthesized on cells by GALNT2 and then imaged (stained) using B3GNT6. GALNT2 is a polypeptide N-acetylgalactosaminyltransferase that transfers GalNAc residues to nascent polypeptides. FIG. 6 shows the result of imaging the cells on which Tn antigens were synthesized. The fluorescence of the Tn antigens can be clearly seen in FIG. 6. The fluorescence indicates that the synthesized Tn antigens are localized in the cytoplasm. This is expected, as the labeling is most likely on nascent polypeptides that have just emerged from ribosomes in the cytoplasm and have open sites for glycosylation. For the purpose of demonstrating T antigen imaging, cells were stained using GCNT1. FIG. 7 shows the result of imaging the C3H/10T1/2 cells using GCNT1. The fluorescence of the T antigens can be clearly seen in FIG. 7, thus confirming that C3H/10T1/2 cells express T antigens.

### Example 2: Glycan Imaging on HUVEC Cells

HUVEC cells were grown in 24-well plate to confluence and then stained for Tn antigens and HS using B3GNT6 and EXT1/2, respectively. EXT1/2 is a heterodimeric HS polymerase of EXT1 and EXT2. Like in Example 1, Tn antigens were not found on HUVEC cells, so Tn antigens were synthesized on the cells using GALNT2 prior to B3GNT6 staining. FIG. 8 shows the result of imaging the cells on which Tn antigens were synthesized. Similar to C3H/10T1/2 cells, the fluorescence indicates that the Tn antigens are localized in the cytoplasm and the regions immediately surrounding the nuclei but not in the nuclei of HUVEC cells.

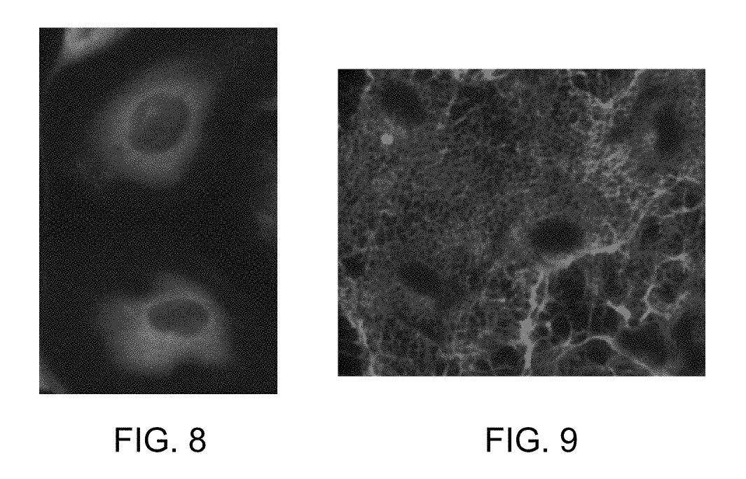

For the purpose of demonstrating HS imaging, the cells were stained using EXT1/2. FIG. 9 shows the result of imaging the cells using EXT1/2. The fluorescence of HS can clearly be seen in the extracellular matrix of the cells. FIG. 9 indicates that there is an abundance of HS in the extracellular matrix as compared to the cell bodies.

### Example 3: Specificity of HS Staining

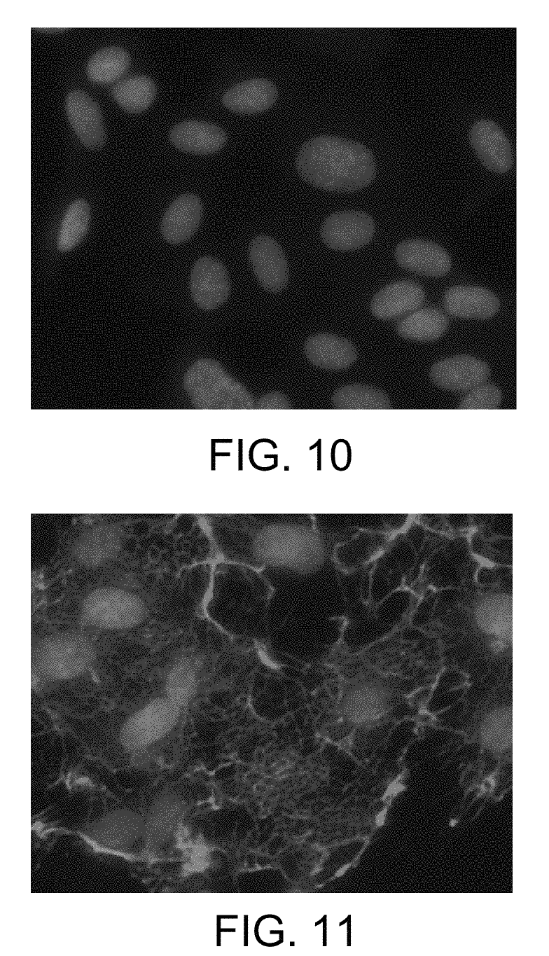

To test the specificity of the HS imaging method, HUVEC cells were treated with Hep III and HPSE enzymes prior to staining with EXT1/2. Hep III digestion of HS results in Δ4,5-glucuronic acid (ΔGlcA) residues at the ends of HS chains. AGlcA lacks a C₄—OH group that is required for HS chain extension by EXT1/2. Therefore Hep III digestion should prevent the labeling of HS with EXT1/2. HUVEC cells digested with Hep III were stained using EXT1/2. The cells were also stained using DAPI in order to show the location of the nuclei. FIG. 10 shows the result of imaging the cells using EXT1/2 and DAPI after digestion with Hep III. As can be seen in FIG. 10, only the fluorescence of the nuclei is apparent. This confirms that the imaging method is specific to HS, since it is known that Hep III prohibits EXT1/2 from labeling HS chains.

HPSE digestion on HS results in uniform N-sulfated GlcNAc residues (GlcNS) at the ends of HS chains. Therefore, pretreatment with HPSE should significantly increase the labeling of HS chains. HUVEC cells digested with HPSE were stained using EXT1/2. The cells were also stained using DAPI in order to show the location of the nuclei. FIG. 11 shows the result of imaging the cells using EXT1/2 and DAPI after digestion with HPSE. As can be seen in FIG. 11, the fluorescence of the HS in the extracellular matrix is apparent. This confirms that the GlcNS residues were extended by EXT1/2.

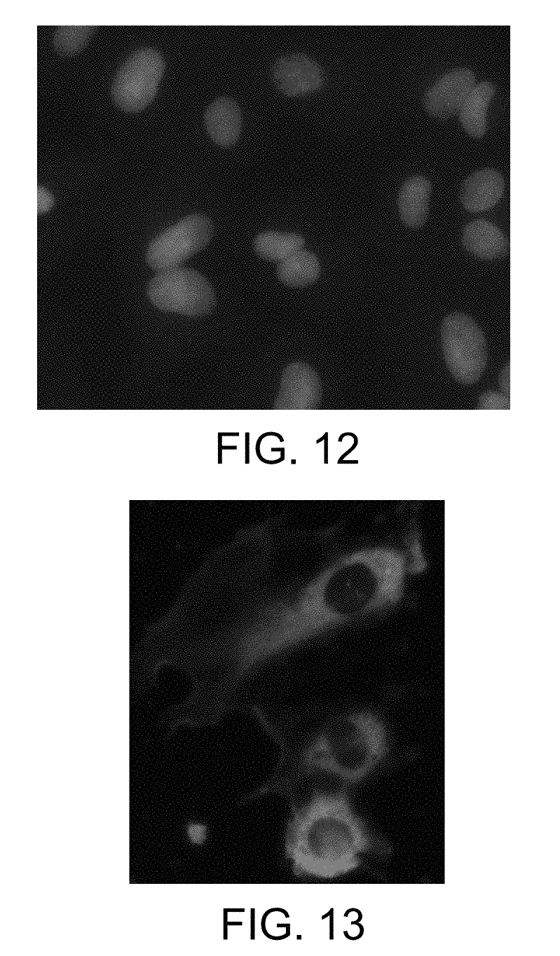

Finally, HPSE treated cells were also labeled using EXT1/2 without the presence of the donor substrate UDP-GlcA as a negative control, as the nascent GlcNS exposed by HPSE can only be labeled with GlcNAz via a GlcA residue. FIG. 12 shows the result of imaging HPSE treated cells using EXT1/2 and DAPI in the absence of UDP-GlcA. As can been seen in FIG. 12, only the fluorescence of the nuclei is apparent. This confirms that GlcA is a prerequisite for the incorporation of GlcNAz into the newly exposed GlcNS residues on HS chains after digestion with HPSE.

### Example 4: Specificity of T Antigen Staining

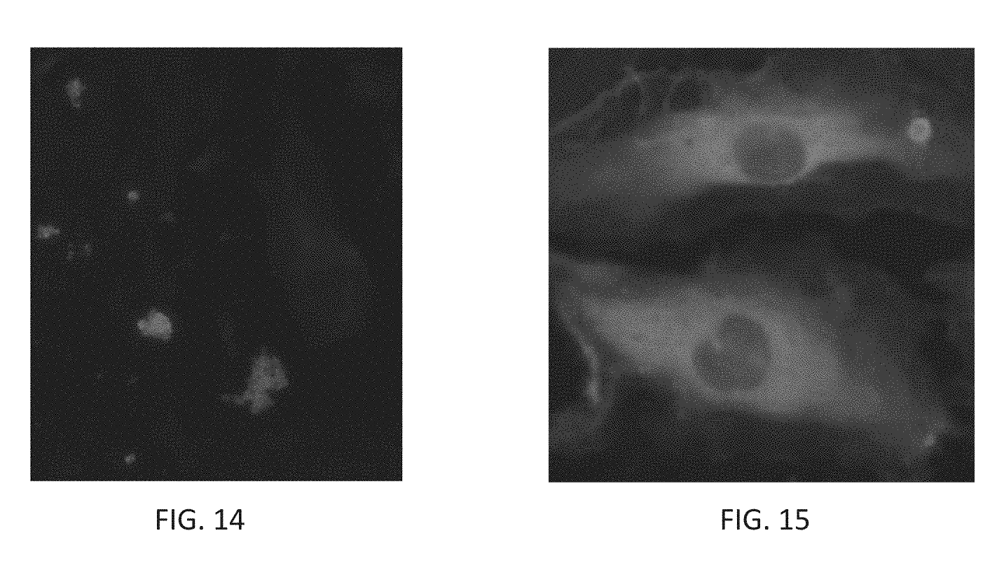

To test the specificity of T antigen staining using GCNT1, T antigens were synthesized using GALNT2 and ClGalT1 on T antigen-free HUVEC cells. GALNT2 attaches a GalNAc residue to serine or threonine residues on a protein backbone to generate a Tn antigen, and ClGalT1 adds a Gal residue to O-GalNAc to complete the T antigen synthesis. The T antigen-free cells were pretreated with both GALNT2 and ClGalT1 and subsequently imaged using GCNT1. FIG. 13 shows the result of imaging the GALNT2 and C1GalT1 treated cells using GCNT1. As shown in FIG. 13, the fluorescence of the T antigens can be clearly seen. T antigen-free HUVEC cells were also pretreated with just GALNT2 to produce Tn antigens and subsequently stained using GCNT1. FIG. 14 shows the result of imaging the GALNT2 treated cells using GCNT1. As shown in FIG. 14, almost no fluorescence is apparent. This confirms the specificity of GCNT1 for labeling T antigens.

### Example 5: Synthesis and Imaging of Tn Antigens on Fixed HUVEC Cells

HUVEC cells were fixed using 4% paraformaldehyde and then incubated with 5 μg recombinant GALNT2 and 5 μg recombinant human B3GNT6 in the presence of 100 μM UDP-GalNAc, 100 uM UDP-GlcNAz, and 10 mM MnCl₂ for 1 hour at 37° C. The incorporated GlcNAZ was further conjugated to a biotin molecule via click chemistry and detected by streptavidin conjugated Alexa Fluor® 555. The image was captured with a ZEISS Axiocam 506 mono camera.

FIG. 15 shows the result of imaging the cells with the synthesized Tn antigen using B3GNT6 . As shown in FIG. 15, the fluorescence of the Tn antigens shows that the Tn antigens are localized in the cytoplasm. This confirms that Tn antigens can be synthesized using GALNT2 in vitro. This also confirms that Tn antigens can be detected and imaged sing B3GNT6.

### Example 6: Synthesis and Imaging of T Antigens on Fixed HUVEC Cells

HUVEC cells in a 24-well plate were fixed using 4% paraformaldehyde and then incubated with 5 μg recombinant GALNT2, 5 μg recombinant human ClGALT1, and 5 μg recombinant human GCNT1 in the presence of 100 μl UDP-GalNAc, 100 μM UDP-Gal, UDP-GlcNAz, and 10 mM MnCl₂ for 1 hour at 37° C. The incorporated GlcNAz was further conjugated to a biotin molecule via click chemistry and detected by streptavidin conjugated Alexa Fluor® 555.

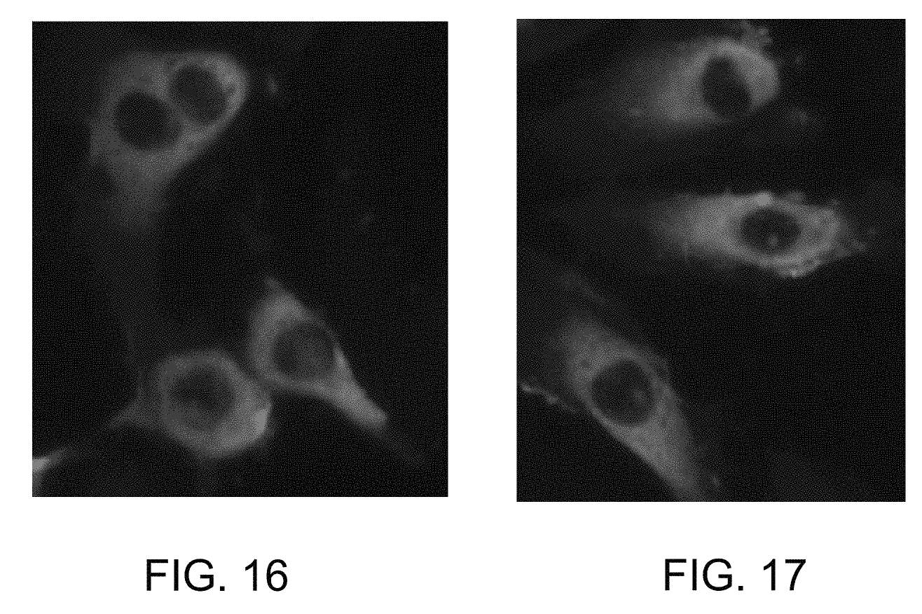

FIG. 16 shows the result of imaging the cells with the synthesized T antigen using GCNT1. As shown in FIG. 16, the fluorescence of the T antigens shows that the T antigens are localized in the cytoplasm. This confirms that T antigens can be synthesized by sequential addition of GalNAc and Gal residues on HUVEC cells in vitro. This also confirms that T antigens can be detected and imaged using GCNT1.

### Example 7: Synthesis and Imaging of Sialylated-T Antigens on Fixed HUVEC Cells

HUVEC cells in a 24-well plate were fixed using 4% paraformaldehyde and then incubated with 5 μg each of recombinant GALNT2 and rhC1GALT1 in the presence of 100 μM each of their donor substrates UDP-GalNAc and UDP-Gal to form T antigens on the cells. Sialylated-T antigens with incorporated azido-sialic acid were subsequently formed by incubating the cells with 5 μg recombinant human ST3Gal1 and CMP-N₃-Neu5Ac (azido-sialic acid). ST3Gal1 attaches azido-sialic acid for purposes of both synthesis and labeling. The incorporated azido-sialic acid was further conjugated to a biotin molecule via click chemistry and detected by streptavidin conjugated Alexa Fluor® 555.

FIG. 17 shows the result of imaging the cells with the synthesized sialylated-T antigen using ST3Gal1. As shown in FIG. 17, the fluorescence of the sialylated-T antigens shows that the sialylated-T antigens are localized in the cytoplasm. This confirms that sialylated-T antigens can be synthesized by sequential addition of GalNAc, Gal, and sialic acid residues on HUVEC cells in vitro.

### Example 8. Imaging Sialylated-T/Tn and T/Tn Antigens on Live HeLa Cells

Live HeLa cells in a 24-well plate were first incubated with 10 μg of recombinant C. perfringens neuraminidase (sialidase) in 200 microliters (μl) of a buffer of 25 mM Tris and 150 mM NaCl at pH 7.5 for 5 minutes at room temperature to remove the terminal sialic acid from sialylated-T and sialylated-Tn antigens. The neuraminidase treated cells were then incubated with 10 μg recombinant ST6GalNAc1 and 100 μM CMP-azido-sialic acid, in 25 mM Tris, 150 mM NaCl, 10 mM MnCl2, at pH 7.5 for 20 minutes at 37° C. to incorporate azido-sialic acid into the antigens on the cells. The cells were then fixed using 4% paraformaldehyde for 10 minutes. The incorporated azido-sialic acid was further conjugated to a biotin molecule via click chemistry and detected by streptavidin conjugated Alexa Fluor® 555. The cells were also stained with DAPI in order to show the location of the nuclei. The image was captured with a ZEISS Axiocam 506 mono camera.

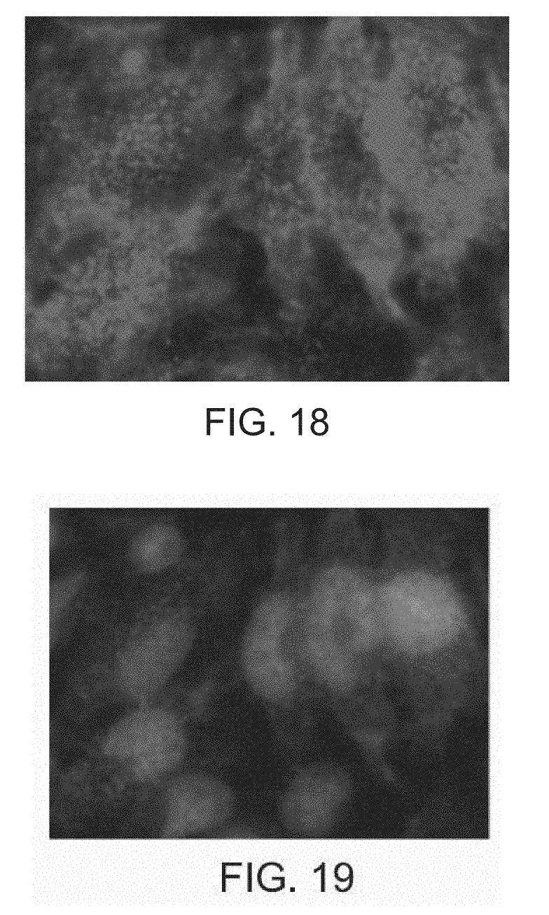

FIG. 18 shows the result of imaging the neuraminidase (sialidase) treated cells using ST6GalNAc1. FIG. 19 shows the result of imaging the neuraminidase (sialidase) treated cells using ST6GalNAc1 and DAPI. As shown in FIGS. 18-19, the fluorescence of the T, Tn, sialylated-T, and sialylated-Tn antigens shows that the antigens are localized in the cytoplasm. This confirms that T, Tn, sialylated-T, and sialylated-Tn antigens can be detected and imaged using ST6GalNAc1 after treatment with neuraminidase (sialidase).

1. An in vitro method of imaging a cancer antigen, the method comprising:
providing a sample comprising a cancer antigen selected from the group consisting of a Tn antigen, a T antigen, a sialylated-Tn antigen, and a sialylated-T antigen;
treating the sample with a glycosyltransferase to incorporate a carbohydrate into the cancer antigen, wherein the carbohydrate includes a click chemistry moiety;
adding a label to the sample, wherein the label includes a click chemistry moiety that reacts to the click chemistry moiety of the carbohydrate such that the label attaches to the carbohydrate to form a labeled cancer antigen; and
imaging the sample with a camera.

2. The method of claim 1, wherein the glycosyltransferase is selected from the group consisting of B3GNT6, GCNT1, ST3Gal1, ST3Gal2, ST6GalNAc1, ST6GalNAc2, and ST6GalNAc4.

3. The method of claim 1, further comprising treating the sample with sialidase prior to treating the sample with the glycosyltransferase to expose a glycosyltransferase recognition site.

4. The method of claim 1, wherein the label is a fluorescent label, a colorimetric label, a biotin linked to a fluorescent label, or a biotin linked to a colorimetric label, and wherein the carbohydrate includes a click chemistry moiety selected from one of an azido group or an alkyne group and the label includes a click chemistry moiety selected from the other of the azido group or the alkyne group.

5. The method of claim 1, wherein the carbohydrate is GlcNAz or azido-sialic acid.

6. An in vitro method of synthesizing and imaging a Tn antigen, the method comprising:
providing a sample comprising a polypeptide chain having a serine or threonine residue;
treating the sample with a first glycosyltransferase to attach a GalNAc residue to the serine or threonine residue;
treating the sample with a second glycosyltransferase to incorporate a carbohydrate into the GalNAc residue, wherein the carbohydrate includes a click chemistry moiety;
adding a label to the sample, wherein the label includes a click chemistry moiety that reacts to the click chemistry moiety of the carbohydrate such that the label attaches to the carbohydrate to form a labeled Tn antigen; and
imaging the sample with a camera.

7. The method of claim 6, wherein the first glycosyltransferase is selected from the group consisting of GALNT1, GALNT2, and GALNT3.

8. The method of claim 6, wherein the second glycosyltransferase is selected from the group consisting of B3GNT6, ST6GalNAc1, and ST6GalNAc2.

9. The method of claim 6, wherein the label is a fluorescent label, a colorimetric label, a biotin linked to a fluorescent label, or a biotin linked to a colorimetric label, and wherein the carbohydrate includes a click chemistry moiety selected from one of an azido group or an alkyne group and the label includes a click chemistry moiety selected from the other of the azido group or the alkyne group.

10. The method of claim 6, wherein the carbohydrate is GlcNAz or azido-sialic acid.

11. An in vitro method of synthesizing and imaging a T antigen, the method comprising:
providing a sample comprising a polypeptide chain having a serine or threonine residue;
treating the sample with a first glycosyltransferase to attach a GalNAc residue to the serine or threonine residue;
treating the sample with a second glycosyltransferase to attach a galactose residue to the GalNAc residue;
treating the sample with a third glycosyltransferase to incorporate a carbohydrate into the GalNAc residue or the galactose residue, wherein the carbohydrate includes a click chemistry moiety;
adding a label to the sample, wherein the label includes a click chemistry moiety that reacts to the click chemistry moiety of the carbohydrate such that the label attaches to the carbohydrate to form a labeled T antigen; and
imaging the sample with a camera.

12. The method of claim 11, wherein the first glycosyltransferase is selected from the group consisting of GALNT1, GALNT2, and GALNT3, and wherein the second glycosyltransferase is C1GalT1.

13. The method of claim 11, wherein the third glycosyltransferase is selected from the group consisting of GCNT1, ST3Gal1, ST3Gal2, ST6GalNAc1, and ST6GalNAc2.

14. The method of claim 11, wherein the label is a fluorescent label, a colorimetric label, a biotin linked to a fluorescent label, or a biotin linked to a colorimetric label, and wherein the carbohydrate includes a click chemistry moiety selected from one of an azido group or an alkyne group and the label includes a click chemistry moiety selected from the other of the azido group or the alkyne group.

15. The method of claim 11, wherein the carbohydrate is GlcNAz or azido-sialic acid.

16. An in vitro method of synthesizing and imaging a sialylated-T antigen, the method comprising:
providing a sample comprising a polypeptide chain having a serine or threonine residue;
treating the sample with a first glycosyltransferase to attach a GalNAc residue to the serine or threonine residue;
treating the sample with a second glycosyltransferase to attach a galactose residue to the GalNAc residue;
treating the sample with a third glycosyltransferase to attach a sialic acid residue to the galactose residue;
treating the sample with a fourth glycosyltransferase to incorporate a carbohydrate into the GalNAc residue, wherein the carbohydrate includes a click chemistry moiety;
adding a label to the sample, wherein the label includes a click chemistry moiety that reacts to the click chemistry moiety of the carbohydrate such that the label attaches to the carbohydrate to form a labeled sialylated-T antigen; and
imaging the sample with a camera.

17. The method of claim 16, wherein the first glycosyltransferase is selected from the group consisting of GALNT1, GALNT2, and GALNT3, wherein the second glycosyltransferase is C1GalT1, and wherein the third glycosyltransferase is selected from the group consisting of ST3Gal1 and ST3Gal2.

18. The method of claim 16, wherein the fourth glycosyltransferase is selected from the group consisting of ST6GalNAc1, ST6GalNAc2, and ST6GalNAc4.

19. The method of claim 16, wherein the label is a fluorescent label, a colorimetric label, a biotin linked to a fluorescent label, or a biotin linked to a colorimetric label, and wherein the carbohydrate includes a click chemistry moiety selected from one of an azido group or an alkyne group and the label includes a click chemistry moiety selected from the other of the azido group or the alkyne group.

20. The method of claim 16, wherein the carbohydrate is azido-sialic acid.

21. A kit for imaging a cancer antigen in vitro, the kit comprising:
a glycosyltransferase specific to a cancer antigen selected from the group consisting of a Tn antigen, a T antigen, a sialylated-Tn antigen, and a sialylated-T antigen;
a donor carbohydrate with a click chemistry moiety;
a label including a click chemistry moiety that reacts to the click chemistry moiety of the donor carbohydrate; and
click chemistry reagents.

22. The kit of claim 21, wherein the glycosyltransferase is selected from the group consisting of B3GNT6, GCNT1, ST3Gal1, ST3Gal2, ST6GalNAc1, ST6GalNAc2, and ST6GalNAc4.

23. The kit of claim 22, wherein the carbohydrate is GlcNAz or azido-sialic acid.

24. The kit of claim 22, wherein the label is a fluorescent label, a colorimetric label, a biotin linked to a fluorescent label, or a biotin linked to a colorimetric label, and wherein the carbohydrate includes a click chemistry moiety selected from one of an azido group or an alkyne group and the label includes a click chemistry moiety selected from the other of the azido group or the alkyne group.

25. An in vitro method of screening a test substance as a therapeutic agent for treating cancer, the method comprising:
providing a sample comprising a cancer cell;
treating the sample with the test substance;
treating the sample with a glycosyltransferase to incorporate a carbohydrate into a cancer antigen selected from the group consisting of a Tn antigen, a T antigen, a sialylated-Tn antigen, and a sialylated-T antigen, wherein the carbohydrate includes a click chemistry moiety;
adding a label to the sample, wherein the label includes a click chemistry moiety that reacts to the click chemistry moiety of the carbohydrate such that the label attaches to the carbohydrate to form a labeled cancer antigen that generates a signal upon imaging;
imaging the sample with a camera to determine the strength of the signal generated by the labeled cancer antigen;
comparing the image of the sample treated with the test substance with an image of an untreated sample comprising a cancer cell with the labeled cancer antigen; and
designating the test substance as a therapeutic agent for treating cancer when the signal generated by the labeled cancer antigen of the sample treated with the test substance is weaker than the signal generated by the labeled cancer antigen in the untreated sample.

26. The method of claim 25, wherein the glycosyltransferase is selected from the group consisting of B3GNT6, GCNT1, ST3Gal1, ST3Gal2, ST6GalNAc1, ST6GalNAc2, and ST6GalNAc4.

27. The method of claim 25, wherein the carbohydrate is GlcNAz or azido-sialic acid.

28. The method of claim 25, wherein the label is a fluorescent label, a colorimetric label, a biotin linked to a fluorescent label, or a biotin linked to a colorimetric label, and wherein the carbohydrate includes a click chemistry moiety selected from one of an azido group or an alkyne group and the label includes a click chemistry moiety selected from the other of the azido group or the alkyne group.
